(19) United States
(12) Patent Application Publication
(10) Pub. No.: US 2024/0174976 A1
(43) Pub. Date: May 30, 2024

(54) BIOINK, MOLDED BODY, ARTICLE, AND METHOD FOR PRODUCING MOLDED BODY

(71) Applicants: NIPPI, INCORPORATED, Tokyo (JP); SUNARROW LTD., Tokyo (JP)

(72) Inventors: Shinomi Yagi, Tokyo (JP); Keisuke Tanaka, Tokyo (JP); Shunji Hattori, Tokyo (JP); Kazunori Mizuno, Tokyo (JP); Masataka Tanaka, Tokyo (JP); Kazuyosi Sato, Tokyo (JP)

(21) Appl. No.: 18/546,519

(22) PCT Filed: Feb. 16, 2022
(86) PCT No.: PCT/JP2022/006095

§ 371 Date: Aug. 15, 2023

(30) Foreign Application Priority Data
Feb. 16, 2021 (JP) 2021-022602

(51) Int. Cl. C12N 5/00; B33Y 10/00; B33Y 70/00; B33Y 80/00

(52) U.S. Cl. CPC C12N 5/0062; B33Y 70/00; B33Y 80/00; B33Y 10/00; C12N 2513/00; C12N 2533/54; C12N 2537/10

(57) ABSTRACT

Provided are a bioink including a collagen fiber with an average fiber length of from 0.5 to 1,000 μm, a shaped object printed with the bioink, a product, and a method for producing a shaped object. The bioink is characterized by including a collagen fiber composed of collagen and/or a collagen derivative and a solvent, the collagen fiber having an average fiber length of from 0.5 to 1,000 μm. The bioink can be extruded from a 3D printer even with a collagen fiber concentration of from 5 to 30% (w/w). The shaped object obtained by bioprinting is excellent in terms of resolution, thermal stability, and structural stability.

### TECHNICAL FIELD

The present disclosure relates to a bioink comprising a collagen fiber with an average fiber length of from 0.5 to 1,000 μm, a shaped object using the bioink, a product such as a cell culture substrate comprising a shaped object or the like, and a method for producing a shaped object.

### BACKGROUND ART

A construct consisting of extracellular matrix (ECM) molecules such as collagen has excellent tissue regeneration ability when filled into a defective site in a living body and can be suitably used as an artificial material for regenerative medicine (Patent Literature 1). However, producing 3D shaped objects is not easy. Patent Literature 1 discloses only a sheet-like product obtained by filtering a material and molding the material into a flat shape and a cubic-formed product obtained by filling a columnar mold with a material to mold the material.

Meanwhile, there is 3D bioprinting as a technique for forming an arbitrary shape of living tissue using a bioink containing ECM molecules such as collagen. By forming layers of a bioink to form a tissue or organ, the resulting tissue or organ can be applied to medical research, such as regenerative medicine and the replacement of functional organs. In addition, it is also possible to produce a 3D shaped object in which cells are deposited using a bioink as a support material. Such a 3D shaped object can be used as a scaffold for cells during in vitro or in vivo culture. Further, alternatives to animal testing that use 3D shaped object to evaluate the effects of cosmetics and pharmaceuticals can also contribute to reducing the use of laboratory animals.

There is, as a collagen-containing bioink, a bioink comprising an undenatured collagen and having a static stiffness of from about 100 to about 150,000 Pa at room temperature and a shear stiffness of less than about 50 Pa at a shear rate of greater than 0.001 sec⁻¹ (Patent Literature 2). The bioink described in Patent Literature 2 was made in view of the conventional problems, that is, the problem that shaped objects of conventional bioinks gel at 37° C. to facilitate cell attachment, but low collagen concentrations can only provide minimal structural integrity to 3D structures, the problem that printed conventional bioinks experience shear-thinning and cannot thus regain stiffness, and the problem that neutralized collagen inks gel in syringes and clog printers. The bioink described in Patent Literature 2 can be mixed with cells at neutral pH, can be printed in a cell culture medium, and has excellent working time and rigidity when printed in a cell culture medium.

### CITATION LIST

### Patent Literature

Patent Literature 1: International Publication No. WO2013/105665
Patent Literature 2: Unexamined Japanese Patent Application Publication (Translation of PCT application) No. 2019-530461.
Patent Literature 3: International Publication No. WO2012/15055

### SUMMARY OF INVENTION

### Technical Problem

Collagen exists extracellularly as a fiber and in vivo constitutes various tissues at high concentrations of 25% in the skin, 32% in tendons, 16% in cartilage, 23% in bones, and 18% in dentin per wet weight. However, a collagen solution has a high viscosity and is difficult to dissolve at a high concentration similar to that in vivo. Patent Literature 2 describes that the collagen concentration may be 50 mg/mL but discloses no example in which the collagen concentration is 50 mg/mL. Meanwhile, in a case in which a 3D shaped object consisting of collagen is used as an organ substitute, it is preferable that the collagen density of the shaped object is close to the collagen density of living tissue. In general, since the collagen density of the 3D shaped object depends on the collagen concentration of the bioink used as a raw material, the development of a bioink with a higher collagen concentration is desired.

Meanwhile, since a collagen solution gels at 37° C. under neutral salt conditions, in a case in which a collagen solution is printed with a 3D printer, printing needs to be performed from 1° C. to 10° C. to avoid clogging due to gelation. A room-temperature printing operation results in a limited working time due to the increase in temperature of the bioink over time. In addition, low collagen concentrations require extrusion into a supporting medium such as a cell culture medium. However, the bioink diffuses into the supporting medium until it gels, which may result in poor resolution of the resulting 3D shaped object. Therefore, it is desired to develop a bioink that can ensure fluidity when extruded from a 3D printer and can be 3D-printed without a supporting medium, such as a cell culture medium.

In view of the above-described circumstances, an objective of the present disclosure is to provide a bioink that can contain a collagen fiber at a high concentration.

Another objective of the present disclosure is to provide a shaped object that is printed using the bioink.

Another objective of the present disclosure is to provide a product such as a cell culture substrate comprising the shaped object.

Another objective of the present disclosure is to provide a method for producing a shaped object using the bioink.

### Solution to Problem

With a focus on the average fiber length of collagen fibers, the present disclosers made findings including that using collagen with an average fiber length shorter than the average fiber length of about 2,000 μm of conventional collagen fibers allows even a bioink with a high collagen concentration to be extruded without clogging the nozzle of a 3D printer, and such a high collagen concentration allows 3D printing by extruding the bioink into the atmosphere without using a supporting medium during bioprinting. This has led to the completion of the present disclosure.

In other words, the present disclosure provides a bioink for use in bioprinting, which comprises collagen fibers composed of collagen and/or a collagen derivative and a solvent, wherein an average fiber length of the collagen fibers is from 0.5 to 1,000 μm.

The present disclosure also provides the bioink, in which the concentration of the collagen fiber in the solvent is from 5 to 30% (w/w).

The present disclosure also discloses the bioink, in which the solvent is one or more selected from the group consisting of deionized water, a buffer, physiological saline, and a cell culture medium.

The present disclosure also provides the bioink, which further contains one or more compounds selected from the group consisting of an extracellular matrix molecule, a decellularized tissue, a growth factor, and a cytokine.

The present disclosure also provides the bioink, which further contains cells.

The present disclosure also provides the bioink, which is a bioink for 3D printer.

The present disclosure also provides a shaped object, which including the bioink.

The present disclosure also provides a crosslinked shaped object, which comprises the shaped object that is crosslinked with one or more selected from the group consisting of riboflavin, methacrylated gelatin (GelMA), polyethylene glycol diacrylate (PEGDA), glutaraldehyde, formaldehyde, genipin, an ammonium derivative, a photoinitiator, Irgacure (registered trademark), lithium phenyl-2,4,6-trimethylbenzoylphosphinate, and ruthenium.

The present disclosure also provides one or more products comprising the shaped object or the crosslinked shaped object, which is selected from the group consisting of a cell culture substrate, a substrate for transplantation, a tissue construct, an organ model, and a substrate for regenerative medicine.

The present disclosure also provides a method for producing a shaped object, characterized by extruding the bioink into the atmosphere by pressurization via a nozzle with a diameter of from 0.2 to 1 mm.

### Advantageous Effects of Invention

According to the present disclosure, a bioink comprising collagen fibers with an average fiber length of from 0.5 to 1,000 μm, a shaped object comprising the bioink, a product comprising the shaped object, and a method for producing a shaped object using the bioink are provided.

### BRIEF DESCRIPTION OF DRAWINGS

FIG. 1 is a diagram illustrating a shaped object prepared in Example 2, in which panel A illustrates a grid-like sheet, panel B illustrates an oriented sheet, panel C illustrates a cube (grid-like), and panel D illustrates a nose model;

FIG. 2 is a diagram illustrating an oriented sheet prepared in Example 2, in which panel A illustrates 30×, 50×, and 100× enlarged views of the surface of the oriented sheet, and panel B illustrates 50×, 100×, and 200× enlarged views of the cross-section of the oriented sheet;

FIG. 3 is a diagram illustrating that the oriented sheet prepared in Example 2 has a fibrous structure (arrow) derived from collagen, in which panel A illustrates the surface of the oriented sheet and panel B illustrates the cross-section of the oriented sheet;

FIG. 4 is a diagram indicating the results of Example 3, in which fibrous structures derived from collagen are indicated by arrows, and a tendency for collagen fibrils to increase with the incubation time was observed;

FIG. 5 is a diagram indicating the results of Example 4, illustrating the turbidity changes of the solution when a collagen solution in which the oriented sheet was dissolved was incubated at 37° C. under neutral salt conditions;

FIG. 6 is a diagram indicating the results of Example 4, illustrating the circular dichroism (CD) spectra of the acetic acid solution of oriented sheet-derived collagen (A), the acetic acid solution of collagen powder (B), and the acetic acid solution of oriented sheet-derived collagen heat-treated at 50° C. (C), and there was a 221-nm peak indicating the undenatured state for the acetic acid solution of oriented sheet-derived collagen and the acetic acid solution of collagen powder-derived collagen, while the peak disappeared due to heat treatment at 50° C., indicating denaturation;

FIG. 7 is a diagram indicating the results of Example 5, in which panel A illustrates the denaturation temperature by a differential scanning calorimeter of the composition in which the oriented sheet prepared in Example 2 was wetted with phosphate-buffered saline (PBS), and panel B illustrates the denaturation temperature by a differential scanning calorimeter of the pepsin-solubilized collagen (PSC) solution;

FIG. 8 is a diagram indicating the results of Example 6, illustrating phase-contrast microscope images, fluorescence microscope images after calcein staining, and merged images on days 3 and 6 of the static culture of fibroblasts on the grid-like sheet prepared in Example 2;

FIG. 9 is a diagram indicating the results of Example 7, illustrating the time course of OD450 of the culture medium during the culture of fibroblasts on the acid soluble collagen coated-dish (A), collagen gel (B), and the oriented sheet prepared in Example 2 (C) with the addition of the Cell Counting Kit reagent;

FIG. 10 is a diagram indicating the results of Example 7, illustrating fluorescence microscope images after calcein staining, and merged images on days 1, 3 and 7 of the static culture of fibroblasts on the acid soluble collagen coated-dish (A), collagen gel (B), and the oriented sheet prepared in Example 2 (C);

FIG. 11 is a diagram indicating the results of Example 8, illustrating shaped products (oriented sheet and cube) obtained by varying the collagen fiber concentration of the bioink and the nozzle diameter;

FIG. 12 is a diagram indicating the results of Comparative Example 2, in which panel A is a top view image and panel B is a side view image;

FIG. 13 is an image of a shaped object obtained in Comparative Example 3;

FIG. 14 is an image of a grid-like sheet produced with a bioink prepared using chicken-derived type II collagen obtained in Example 10;

FIG. 15 is a diagram indicating the results of Example 11, illustrating images of sheets printed with various bioinks;

FIG. 16 is a diagram illustrating a state in which a cell-laden bioink prepared in Example 12 was extruded via an 18 G needle, and the extruded cell-laden bioink was immersed in a DMEM containing 10% fetal bovine serum (FBS);

FIG. 17 is a diagram indicating the results of Example 12, in which fluorescence microscope images after calcein staining and those after propidium iodide staining on days 2, 4, 8, and 11 of the static culture of the cell-laden bioink comprising fibroblasts and having a collagen fiber concentration of 10% (w/w) prepared in Example 12 and their merged images with the bright field image are illustrated;

FIG. 18 is a diagram indicating the results of Example 13, in which fluorescence microscope images after calcein staining and those after propidium iodide staining on day 4 of the static culture of the cell-laden bioink comprising fibroblasts and having a collagen fiber concentration of 15% (w/w) prepared in Example 13 and their merged images with the bright field image are illustrated; and

FIG. 19 is a diagram indicating the results of Example 14, in which fluorescence microscope images after calcein staining and those after propidium iodide staining on day 4 of the static culture of the cell-laden bioink comprising myoblasts and having a collagen fiber concentration of 10% (w/w) prepared in Example 14 and their merged images with the bright field image are illustrated.

### DESCRIPTION OF EMBODIMENTS

Embodiment 1 of the present disclosure is a bioink for use in bioprinting, which comprises collagen fibers composed of collagen and/or a collagen derivative and a solvent, and which is characterized in that the average fiber length of the collagen fibers is from 0.5 to 1,000 μm.

In the present disclosure, bioprinting means a technique of producing a shaped object using a biomaterial by 3D printer technology. The term “bioink” refers to a raw material for bioprinting. The term “3D printer” refers to a machine that forms a shaped object based on a three-dimensional digital model.

The bioink according to the present disclosure is characterized by comprising collagen fibers composed of collagen and/or a collagen derivative as a biomaterial. Collagen is one of the proteins that mainly constitute the dermis, ligaments, tendons, bones, cartilage, and various internal organs of vertebrates, and is also contained in crustaceans, mollusks, and the like. Collagen used in the present disclosure may be derived from any tissue or organ of any animal. Collagen is classified into type I, type II, and the like in the order of discovery, but any of them may be used. The method for preparing collagen is also not limited. Collagen is mostly insoluble in a living body, but soluble collagens originally contained in a living body may be extracted and used. An extract obtained by solubilizing a tissue containing insoluble collagens by adding an enzyme such as protease, an acid, an alkali, and/or a neutral salt may also be used. To isolate collagen from a collagen-solubilized solution, the salting-out method, the isoelectric precipitation method, and the like are generally used, but any method may be used. Meanwhile, collagen used in the present disclosure has a triple helical structure in which three polypeptides having a collagen-like sequence represented by -(Gly-X-Y)n-, where X and Y represent amino acid residues, are helically assembled. The collagen may be atelocollagen without telopeptides at both ends or telocollagen containing telopeptides, as long as it has a triple helical structure. When the collagen is solubilized by alkali treatment, for example, asparagine residues and glutamine residues may be converted to aspartic acid residues and glutamic acid residues by deamidation, respectively. In addition, amino acids contained in the collagen may be chemically modified. The collagen is not limited to an extract from an animal tissue and may be purified from collagen-highly expressing cells using a known technique or may be produced as a recombinant protein. For example, the collagen may be produced by CHO cells or tobacco cells using gene recombination technology.

The term “collagen derivative” according to the present disclosure refers to a collagen derivative obtained by modifying amino acids constituting the collagen with a different functional group. Examples thereof include acylated collagen and esterified collagen. One example is a collagen derivative obtained by acylating or esterifying collagen after isolated from a tissue. Meanwhile, the collagen may be collagen whose functional group is esterified or acylated during salting-out or isoelectric precipitation of collagen. For example, when extracting a collagen from a collagen-containing tissue, after acylating collagen is prepared in the tissue, the acylated collagen may be solubilized. Similarly, after esterifying an insoluble collagen contained in a collagen-containing tissue, the esterified collagen may be solubilized. The collagen derivative may be acylated or esterified in the collagen extraction process.

The acylated collagen includes such as succinylated collagen, phthalated collagen, and maleylated collagen. Examples thereof include acylated collagen such as succinylated collagen, phthalated collagen, and maleylated collagen, which are obtained by adjusting the pH of an atelocollagen solution extracted by enzymatic treatment to from 9 to 12 and adding an acid anhydride such as succinic acid, phthalic anhydride, or maleic anhydride. Examples of the esterified collagen include a solubilized collagen that has been esterified as well as an esterified collagen that has been solubilized by enzymatic treatment after esterifying an insoluble collagen or the like. An alcohol for obtaining an esterified collagen by acting on collagen may be a primary alcohol, a secondary alcohol, or a tertiary alcohol. The alcohol is not limited to a monohydric alcohol and may be a dihydric alcohol, a trihydric alcohol, or any of the other polyhydric alcohols.

The collagen fibers composed of collagen and/or a collagen derivative used in the present disclosure are characterized by having an average fiber length of from 0.5 to 1,000 μm. The average fiber length is preferably from 10 to 700 μm, more preferably from 100 to 500 μm, particularly preferably from 100 to 250 μm. A fibrillar collagen is formed by which a plurality of collagen molecules assembled with a gap of 67 nm in a neutral aqueous solution to form collagen fibrils, and further, a plurality of collagen fibrils assembled to form a collagen fiber. When a salt is added to a collagen solution, the collagen is precipitated by salting-out. In addition, when an acid or alkali is added to adjust the pH to near the isoelectric point of collagen, the collagen is isoelectrically precipitated. When the collagen precipitate thus formed is dissolved in an aqueous solution at 37° C. under neutral salt conditions, collagen fibrils and fibers are formed by the association of collagen molecules. In general, the formed collagen fibers have an average fiber length of about 2,000 μm, considering the balance with the solubility. Meanwhile, collagen fibers with an average fiber length of 0.5 to 1,000 μm can be prepared by reducing the association of collagen molecules in the solution or by physically cutting fibers formed by the association. Collagen fibers with an average fiber length adjusted in such a manner can be used in the present disclosure. The method for adjusting the collagen fiber length in such a manner may be, for example, a method for adjusting the average fiber length according to the description of Patent Literature 3. For example, a predetermined amount of an alkali-solubilized collagen precipitate obtained by solubilization treatment on the dermis layer of porcine skin and salting-out is dispersed in distilled water, adjusted to pH 4.5, and stirred with a stone mill grinder such as a masscolloider to crush collagen fibers, thereby obtaining an isoelectric precipitate. When the solution is stirred with a stone mill grinder or the like and the precipitate is ground and crushed during the formation of the collagen precipitate, the intense stirring inhibits the association of the collagen molecules, and the crushing cuts off the assembled collagen fibers. By adjusting the degree of stirring and crushing, collagen fibers with an average fiber length of from 0.5 to 1,000 μm can be produced.

Meanwhile, Non-fibrillar collagen such as type IV collagen differs from fibrillar collagen in forming a fine mesh-like structure. However, similar to fibrillar collagen, when a salt is added to the collagen solution, the collagen is precipitated by salting-out, and when an acid or alkali is added to adjust the pH to the isoelectric point range, the collagen is isoelectrically precipitated. When producing a collagen precipitate, the solution is stirred with a stone mill grinder, such as a masscolloider, and the precipitate is ground and crushed such that collagen fibers with an average fiber length of from 0.5 to 1,000 μm can be produced.

In the present disclosure, the collagen fiber length shall be measured by the method shown in the present embodiments below. Specifically, scanning electron microscopy is used for dried collagen fibers, and phase-contrast microscopy is used when measuring using a liquid in which collagen fibers are dispersed. The fiber lengths of 20 or more collagen fibers randomly selected are measured, and the value calculated as the average is taken as the average fiber length of the collagen fibers.

A collagen fiber used herein may be a commercially available product. For example, a product with a collagen average fiber length of from 0.5 to 1,000 μm may be selected from commercially available products such as the pepsin-solubilized collagen (PSC) powder manufactured by Nippi, Incorporated and the acid soluble collagen (ASC) powder manufactured by Nippi, Incorporated for use. Specific examples include bovine dermis-derived type I collagen powder (pepsin-solubilized) PSC-1-100-500PW and bovine dermis-derived type I collagen powder (acid soluble) ASC-1-100-500PW.

As described above, a collagen molecule has a triple helical structure, and a collagen molecule having this structure is referred to as “undenatured collagen.” Since a collagen fiber comprises undenatured collagen, according to the bioink of the present disclosure, a shaped product formed with undenatured collagen can be prepared. Even in a case in which the resulting shaped product is wetted in a cell culture medium or the like, the collagen fiber structure comprising undenatured collagen can be maintained in the shaped object.

A wide range of solvents, with which collagen fibers with an average fiber length of from 0.5 to 1,000 μm can be uniformly dissolved, dispersed, and mixed, can be used as a solvent that is a component of the bioink. The pH of the solvent is preferably from 5.0 to 9.0. For example, deionized water, buffer solutions such as phosphate-buffered saline and Tris buffer, physiological saline, cell culture media, and the like can be suitably used.

The collagen fiber concentration in the bioink is from 5 to 30% (w/w). The collagen fiber concentration can be appropriately selected depending on the intended use of the shaped object. To produce a shaped object with high resolution, the collagen fiber concentration is preferably from 10 to 30% (w/w), more preferably from 10 to 25% (w/w). Meanwhile, a shaped object can be produced within a collagen fiber concentration range of 5 to 30% (w/w) depending on the intended use as long as the resolution of the shaped object does not affect the use.

A collagen fiber is not necessarily dissolved in the solvent but only need to be uniformly dispersed in the solvent in the bioink of the present disclosure. As long as the average fiber length of the collagen fibers is from 0.5 to 1,000 μm, the collagen fiber can be uniformly dispersed in the solvent, and the clogging of the nozzle of a 3D printer can be avoided.

The bioink of the present disclosure may further contain the following as long as the properties of the bioink and the resulting shaped object are not impaired: extracellular matrix molecules such as proteoglycan, hyaluronic acid, fibronectin, laminin, tenascin, elastin, fibrillin, and glycosaminoglycan; decellularized tissue; growth factors such as EGF, IGF, TGF, bFGF, NGF, BDNF, VEGF, G-CSF, GM-CSF, PDGF, EPO, TPO, and HGF; and cytokines such as chemokines, interferons, interleukins, and lymphokines. The compounded amount of an extracellular matrix, a cytokine, or a growth factor in the bioink is from 0.1 ng/mL to 10 mg/mL, preferably from 0.1 ng/mL to 1 mg/mL, more preferably from 1 ng/mL to 100 ng/mL. Extracellular matrices and growth factors can induce physiological effects such as differentiation and proliferation specific to cells derived from the tissue. For example, cell differentiation, proliferation, migration, and the like can be regulated by the addition of growth factors. In a case in which a decellularized tissue is added, it can be added in a range of from 0.5 to 2 times the weight of the collagen, more preferably from 0.7 to 1.2 times the weight of the collagen. As long as the amount of the decellularized tissue is less than that of the bioink, the collagen or collagen derivative has little effect on the printing properties.

The bioink of the present disclosure may comprise: inorganic salts such as hydroxyapatite and tricalcium phosphate; synthetic polymers such as polyglycolic acid, polylactic acid, poly(lactide-co-glycolide) copolymer, polydioxanone, poly(methyl acrylate), and poly(methyl methacrylate; nanomaterials such as gold nanoparticles, silver nanoparticles, iron oxide nanoparticles, silica nanoparticles, and carbon nanoparticles; various nutritional components; fluorescent labeling compounds such as a fluorescein derivative, a rhodamine derivative, and a Cy dye; and cross-linkers such as riboflavin, GelMA, PEGDA, glutaraldehyde, formaldehyde, genipin, an ammonium derivative, a photoinitiator, Irgacure (registered trademark), lithium phenyl-2,4,6-trimethylbenzoylphosphinate, and ruthenium. Inorganic substances contribute to increasing the rigidity of the bioink. Mixing metal-based nanoparticles contributes to enhancing electrical conductivity. In addition, a crosslinked product can be prepared by compounding a cross-linker. In the case of using hydroxyapatite in combination, hydroxyapatite is used preferably at a concentration not more than the dissolution concentration in water, which is 0.3 mg/mL.

The bioink may further comprise chemical materials such as nylon or polypropylene and a biological material such as an enzyme, spore, or mycelium. A shaped object containing the above can be produced.

The bioink may further comprise cells, including epithelial cells, endothelial cells, fibroblasts, cardiomyocytes, hepatocytes, smooth muscle cells, skeletal muscle cells, muscle satellite cells, neural Schwann cells, adipocytes, various stem cells such as mesenchymal stem cells, hematopoietic stem cells, hepatic stem cells, epithelial stem cells, germ stem cells, and neural stem cells, as well as pluripotent stem cells such as ES cells and iPS cells. A cell-embedded 3D shaped object can be produced using such a cell-laden bioink.

There is no limit to the method for preparing a bioink. For example, in the case of using a dried collagen fiber such as a collagen powder including collagen fibers with an average fiber length of from 0.5 to 1,000 μm, a bioink can be prepared by uniformly mixing the dried collagen fiber with a solvent. The pH of the solvent is preferably from 5.0 to 9.0, more preferably from 6.0 to 8.0. Mixing can be carried out in a temperature range of from 4° C. to 37° C., preferably at a room temperature of 25° C. The mixing time may be in a range of from 30 seconds to 10 minutes, preferably from 30 seconds to 5 minutes, more preferably from 1 minute to 2 minutes.

In addition, when preparing, as a hydrated collagen fiber, collagen fibers with an average fiber length of from 0.5 to 1,000 μm from an animal tissue, a collagen precipitate obtained by salting-out or isoelectric precipitation can be used as a collagen fiber. The collagen precipitate can be concentrated by centrifugation or diluted by adding various solvents, such as deionized water, to have a desired adjusted collagen fiber concentration. Then, it can be used as a bioink as it is.

Since collagen fibers are highly hydrophilic and viscous, air bubbles may be mixed with the solvent during mixing. A paste-like collagen precipitate obtained by concentrating a salted-out substance or an isoelectric precipitate may also contain air bubbles. Since air bubbles interfere with printing by a 3D printer, it is preferable to perform defoaming after mixing. Examples of the defoaming method include, but are not limited to, ultrasonic defoaming, vacuum depressurization defoaming, and centrifugal defoaming. The defoaming time can be appropriately selected depending on the equipment and method used, and is usually from 30 seconds to 10 minutes, preferably from 30 seconds to 5 minutes, more preferably from 1 minute to 2 minutes. The bioink can be made more homogeneous by mixing, and optionally defoaming and then passing the bioink through a needle with an inner diameter of from 0.3 mm to 1 mm. In a case in which the bioink comprises components other than a collagen fiber, such components may be mixed with the collagen fiber and then mixed and defoamed as described above. In addition, a desired bioink may be prepared by preparing a bioink comprising a collagen fiber in advance and then mixing a solvent comprising other components with the bioink.

The bioink thus prepared is in the paste form in which a collagen fiber and a solvent are uniformly mixed, and as mentioned in the present embodiments described later, the average fiber length of the contained collagen fibers is stably maintained from 0.5 to 1,000 μm.

Meanwhile, the collagen fiber in the bioink is in the form of undenatured collagen. Undenatured collagen may be denatured by heating, pH fluctuations, or enzymatic actions such that the triple helical structure may be destroyed. Therefore, the bioink of the present disclosure is preferably stored at pH 5.0 to 9.0 and 4° C. Low-temperature storage can suppress the deterioration of properties due to collagen fibril formation and prevent denaturation.

The bioink can be loaded into a 3D printer upon bioprinting to be used for producing a shaped object. The 3D printer may be an inkjet 3D printer in which a bioink is made into fine droplets, expelled, and formed into layers for printing or a dispensing 3D printer in which a bioink is extruded for printing. Since the inkjet 3D printer expels a bioink in the form of fine droplets, it is preferable to use a bioink that has a low viscosity and instantly solidifies after printed on the formed surface. Meanwhile, the dispensing 3D printer can use a bioink having a higher viscosity than the inkjet 3D printer. The bioink of the present disclosure has a high collagen fiber concentration and can be suitably used in dispensing 3D printers.

Bioprinting can be carried out by increasing the nozzle diameter of a 3D printer, but the resolution decreases. According to the present disclosure, a shaped object with excellent resolution can be rapidly produced by specifying the average fiber length and the collagen fiber concentration within the above-described ranges. The resolution according to the present disclosure is obtained by, for example, preparing a grid-like sheet as a shaped object, photographing the grid-like sheet from above, selecting arbitrary lines constituting the grid-like sheet, measuring the line widths at five or more sites, and calculating the average of measurements. The resolution can be similarly measured with a grid-like cube instead of the grid-like sheet.

Embodiment 2 of the present disclosure is a shaped object including the bioink. The shaped object may be a sheet-like product, a cubic product, a different irregular-shaped product, or the like formed by extruding one or more layers of a bioink from a nozzle onto a flat surface. This shaped object may be an undried shaped object extruded from a 3D printer, or may be a dried product that has been dehydrated thereafter. The collagen density of a dried shaped object varies depending on the collagen fiber concentration of the bioink used or the shape of the shaped object and is usually from 0.1 to 0.7 g/cm². The inside of the dried product obtained by removing moisture from the printed product by freeze-drying or the like becomes sponge form. Although the drying method is not limited, freeze-drying provides a shaped object in which a collagen fiber is maintained in an undenatured state. The shaped object of the present disclosure is superior in thermal stability to the collagen solution as mentioned in the present embodiments below. In particular, the dried shaped object is excellent in storage stability. The resolution of the dried shaped object depends on the nozzle diameter of the 3D printer, but is from 200 to 1,200 μm.

In a case in which the bioink comprises a cross-linker such as riboflavin, GelMA, PEGDA, glutaraldehyde, formaldehyde, genipin, an ammonium derivative, a photoinitiator, Irgacure (registered trademark), lithium phenyl-2,4,6-trimethylbenzoylphosphinate, or ruthenium, a crosslinked structure can be formed depending on the cross-linker during or after bioprinting. Therefore, the shaped object of the present disclosure may be a crosslinked shaped object as described below. For example, a crosslinked structure is formed by irradiating light during bioprinting, or a crosslinked structure is formed by irradiating an undried shaped object with light after the bioprinting. Light such as ultraviolet light, visible light, or infrared light is applied depending on the cross-linker. The collagen fiber may be cross-linked by light, heat, dehydration, or the like depending on the cross-linker.

Embodiment 3 of the present disclosure is one or more products comprising the shaped object or the crosslinked shaped object, which is selected from the group consisting of a cell culture substrate, a substrate for transplantation, a tissue construct, an organ model, and a substrate for regenerative medicine.

Since the shaped object of the present disclosure comprises collagen fibers, for example, in a case in which the shaped object is a dried product, the dried product is immersed in a solution at pH 5.0 to 9.0, heated to 37° C., and wetted, and then, cultured with cells in a cell culture medium. Accordingly, the cells grow in the shaped object using the collagen fiber as a scaffold. Therefore, the shaped object can be suitably used for a cell culture substrate. By allowing the shaped object to comprise cells and other components, the shaped object can be used as a tissue substitute, such as a substrate for transplantation or regenerative medicine. In addition, by creating a tissue construct similar to that of a living body, it can be used as an in vitro evaluation system for drug screening and the like. The bioink of the present disclosure comprises an undenatured collagen fiber similar to one in a living body and has a high collagen fiber concentration. Therefore, the bioink is also suitable as a substitute for partially lost tissue. In particular, as mentioned in the present embodiments described later, when cells are cultured using a shaped object, the cells grow moderately and are excellent in tissue compatibility. In addition, it was found that when the shaped object is immersed in a cell culture medium, part of the shaped object dissolves over time, but collagen fibrils are reconstituted. When the reconstitution of collagen fiber occurs in the cell culture medium, the formed collagen fibers become long fibers with a fiber length of 2,000 μm or more, and have excellent biocompatibility and stable structure.

The shaped object can be suitably used as a cell culture substrate even when undried. For example, an undried shaped object is immersed in a medium solution or the like to be used, and the solvent contained in the shaped object is replaced with the medium solution. When cells are seeded thereon, the shaped object can function as a cell scaffold material. Since no freeze-drying step is required, the advantage is that it can be applied to cell culture immediately after printing.

Cells that can be cultured in the shaped object of the present disclosure include cells derived from mammals such as humans, mice, rats, cows, and pigs. Examples thereof include epithelial cells, endothelial cells, fibroblasts, cardiomyocytes, hepatocytes, smooth muscle cells, skeletal muscle cells, muscle satellite cells, neural Schwann cells, and adipocytes. The shaped object is also suitable for culturing various stem cells such as mesenchymal stem cells, hematopoietic stem cells, hepatic stem cells, epithelial stem cells, germ stem cells, and neural stem cells, as well as pluripotent stem cells such as ES cells and iPS cells.

The shaped object of the present disclosure can be used as a substitute for tissue in bone grafting and other applications. In this case, an undried shaped object or a dried shaped object can be directly filled into the defect site and used as a substitute, or a shaped object, after culturing cells in advance, can also be used as a substitute. Since the shaped object of the present disclosure functions as a scaffold for cell culture when embedded in a living body, it can also be used as a device for guiding tissue regeneration.

In addition, by mixing cells with the bioink in advance and producing a shaped object with a 3D printer, a tissue construct that mimics the tissue structure of a living body can be prepared. For example, a tissue construct composed of a plurality of cells can be formed by a multi-nozzle 3D printer for efficiently arranging various cells in desired parts of the shaped object. Such a tissue construct can be used, for example, in in vitro metabolism tests in the case of a liver tissue construct, and in skin irritation or eye irritation tests of cosmetics and the like in the case of a skin tissue or corneal tissue construct. In addition, by creating tissue constructs mimicking various organs, it is possible to apply them to drug screening in the field of drug discovery, for example. These are attracting attention as alternatives to animal testing.

In a case in which the shaped object of the present disclosure is printed into the shape of a predetermined organ, the obtained shaped object can also be used as an organ model. Such an organ model can be used, for example, as an optimal training tool for improving surgical techniques using an endoscope. By using an organ model that is most specialized for the texture of each organ, it is possible to perform training that emphasizes tactile impressions. In particular, the organ model can be applied for an organ model for training using thoracic and abdominal cavity simulators and surgical training, a gastric cancer resection (D2 dissection) model, a gastric reconstruction model, a partial nephrectomy model, inguinal hernia surgery model (TAPP), a mitral valve surgery model, a cholecystectomy model, a nerve-sparing dissection model for rectal cancer, lobectomy model, a release sheet, vascular vessels for microsurgery training, and the like. To ensure the rigidity of the shaped object used as the organ model, the obtained shaped object can be crosslinked to have a desired adjusted rigidity.

Embodiment 4 of the present disclosure is a method for producing a shaped object, characterized by extruding the bioink into the atmosphere by pressurization via a nozzle with a diameter of from 0.2 to 1 mm. The bioink of the present disclosure is used for bioprinting, thereby producing a product having a predetermined shape using a 3D printer. Therefore, there is essentially no limit to the nozzle diameter. However, since the average fiber length of collagens used in the bioink of the present disclosure is as short as 0.5 to 1,000 μm, bioprinting can be carried out by extruding the bioink from a nozzle with a diameter of preferably from 0.2 to 1 mm, more preferably from 0.3 to 0.8 mm using a 3D printer even when the collagen fiber concentration is from 5 to 30% (w/w). The viscosity of the bioink increases with an increase in the collagen fiber concentration. Therefore, it is not easy to extrude a 5 to 30% (w/w) bioink via a nozzle with a diameter of from 0.2 to 1 mm. However, since the average fiber length is as short as 0.5 to 1,000 μm, it is possible to extrude the bioink via a nozzle having the above-described diameter, thereby producing a high-resolution shaped object. In addition, since the collagen fiber concentration is as high as 5 to 30% (w/w), it is possible to form a shaped object by layer formation by extruding the bioink into the atmosphere by pressurization via a nozzle without extruding the bioink into the supporting medium. In the case of low collagen concentrations, printing cannot be carried out even with extruding the bioink into the supporting medium unless the nozzle diameter is from 0.1 to 0.3 mm. According to the present disclosure, a shaped object can be produced by extruding a bioink into the atmosphere via a nozzle having a nozzle diameter of more than 0.2 mm. Therefore, a large shaped product can be efficiently produced in a short time without using a supporting medium.

When producing a shaped object using the bioink of the present disclosure, the bioink may be extruded via a nozzle with a diameter of more than 1 mm for printing. Although the resolution is low, the bioink is excellent because a large shaped object can be formed in a short period. In addition, the bioink may be extruded into a supporting medium not limited to the atmosphere for printing. Since the bioink of the present disclosure has a collagen fiber concentration of from 5 to 30% (w/w), the bioink is hardly dispersed even when extruded into a supporting medium, and a shaped object with excellent resolution can be produced. Usually, when a low-concentration collagen solution is used as a bioink, the shaped object may be heated to 37° C. after printing to form gel for increasing rigidity. The bioink of the present disclosure may similarly be subjected to neutral salt conditions after printing and heated at 37° C. for fibril formation treatment. Note that since the collagen fiber concentration in the bioink is high, the rigidity of the shaped object can be maintained without fibril formation treatment, and the reduction in resolution can be suppressed, which is advantageous.

After printing with the 3D printer, the shaped object may be used as is or dehydrated so as to be used as a dried product. The dehydration method can be appropriately selected according to the shape of the shaped object and includes freeze-drying, heat drying, vacuum drying, infrared drying, air drying, and the like. Freeze-drying is suitable for keeping the collagen fiber undenatured.

In a case in which the bioink contains a cross-linker, a cross-linking step may be included in printing with the 3D printer. Alternatively, a cross-linking step may be performed after printing with the 3D printer. For instance, since genipin, a natural cross-linker, is known to have low toxicity to cells, it can be mixed with a bioink and used directly in cell culture without washing or removal.

### EXAMPLES

Next, the present disclosure is specifically described with reference to examples, but these examples do not limit the present disclosure in any way.

### Example 1

Two grams (2 g) of a collagen powder with an average fiber length of 142 μm fractionated with a 100- to 250-μm sieve (PSC powder manufactured by Nippi, Incorporated) and 8 g of deionized water cooled to 4° C. were mixed with a planetary centrifugal mixer (Awatori Rentaro manufactured by THINKY CORPORATION) at 25° C. for 1 minute, followed by defoaming treatment for 1 minute with the mixer. Thus, a bioink with a collagen fiber concentration of 20% (w/w) was prepared. The average fiber length of the collagen fibers contained in the prepared bioink was measured, resulting in 150 μm. The average fiber length of collagen fibers constituting the collagen powder was obtained by randomly selecting 20 or more collagen fibers with a scanning electron microscope, measuring the fiber length, and calculating the average of 20 or more collagen fibers. Meanwhile, the average fiber length after bioink preparation was obtained by taking a portion of the bioink, dispersing in a 50 mM Tris-HCl buffer (pH 8.0), randomly selecting 20 or more collagen fibers with a phase contrast microscope, measuring the fiber length, and calculating the average of 20 or more collagen fibers.

### Example 2

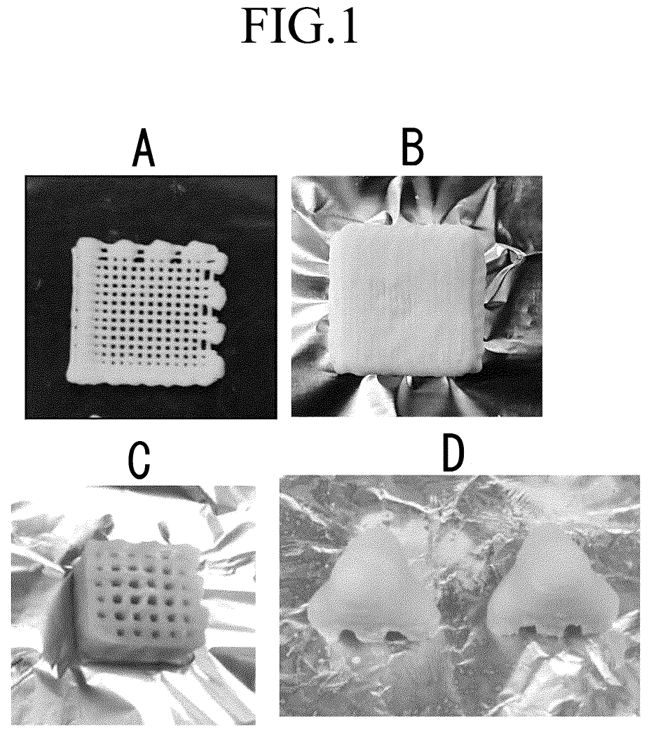

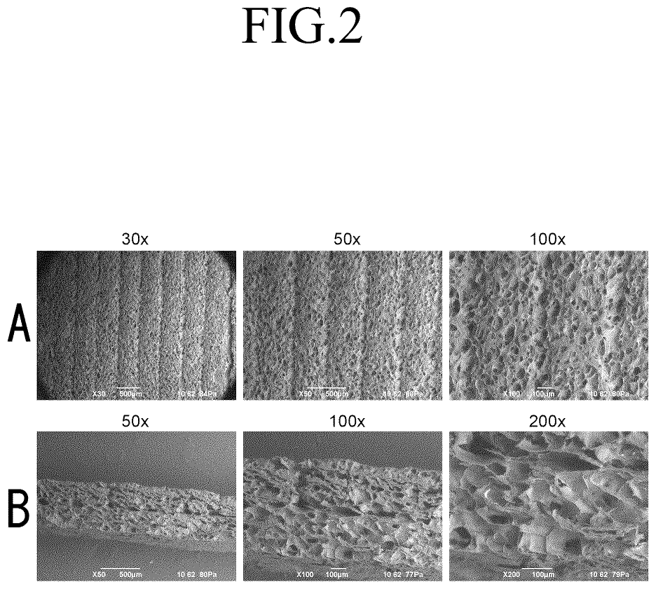

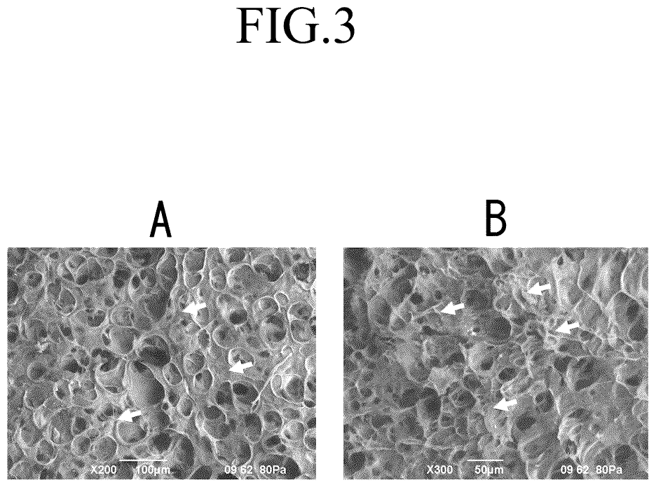

The bioink (at 25° C.) prepared in Example 1 was extruded by applying air at a pressure of from 50 to 300 kPa via a nozzle with a diameter of 0.4 mm from a dispensing 3D printer (Musashi Engineering, Inc., SHOT mini, Model M22-123) into the atmosphere, thereby printing a grid-like sheet 3 cm×3 cm×0.1 cm in size and an oriented sheet 3 cm×3 cm×0.1 cm in size. In addition, a bioink was prepared by the same operation as in Example 1, except that a porcine dermis-derived alkali-solubilized collagen powder with a collagen fiber length of about 158 μm was used instead of the collagen powder (PSC powder manufactured by Nippi, Incorporated) used in Example 1. This bioink was used to perform the same operation as above, thereby printing a grid-like cube 1.7 cm×1.7 cm×1 cm in size and a nose model cube 2.5 cm×3 cm×2.5 cm in size. The time required for printing the grid-like sheet was 2 minutes, the time required for printing the oriented sheet was 5 minutes, the time required for printing the grid-like cube was 10 minutes, and the time required for printing the nose model cube was 30 minutes. Freeze-drying was performed after printing, thereby obtaining a shaped object. The oriented sheet was obtained by extruding the bioink in parallel in a specific direction to form a flat surface and extruding the bioink in parallel in a direction intersecting the direction mentioned above on this flat surface, thereby printing layers. FIG. 1 illustrates the dried shaped objects. Panel A illustrates a grid-like sheet, panel B illustrates an oriented sheet, panel C illustrates a cube (grid-like), and panel D illustrates a nose model shaped object. FIG. 2 illustrates the results of imaging the surface and cross-section of the oriented sheet of B with a scanning electron microscope. Panel A illustrates 30×, 50×, and 100× enlarged views of the surface of the oriented sheet, and panel B illustrates 50×, 100×, and 200× enlarged views of the longitudinal section of the oriented sheet. As illustrated in FIG. 2, uniform orientation in the extrusion direction was observed on the surface of the oriented sheet. The longitudinal section structure of the oriented sheet was in the form of sponge forming uniform cavities. FIG. 3 illustrates an enlarged image of the surface and longitudinal section of the oriented sheet. As illustrated in FIG. 3, a fibrous structure derived from collagen was observed on the surface and in the longitudinal section. Fibrous structures are indicated by arrows.

### Example 3

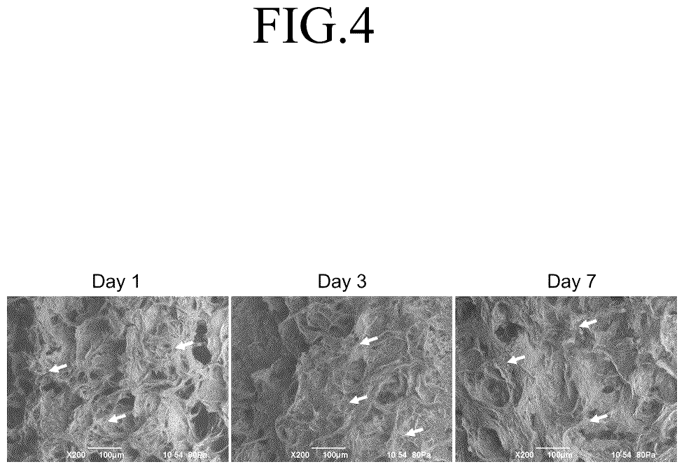

The oriented sheet prepared in Example 2 was punched out with a DERMA-PUNCH (registered trademark) with a diameter of 6 mm, thereby preparing a disc. The disc was wetted in 2 mL DMEM and incubated at 37° C. for 7 days. The disc was recovered on days 1, 3, and 7 after incubation, immobilized in PBS containing 2.5% glutaraldehyde, washed with distilled water, and freeze-dried. Scanning electron microscope images of the disk surface are illustrated in FIG. 4. It was found that the fibrous structure indicated by each arrow was maintained even after 7 days incubation. In addition, a tendency for collagen fibrils to increase with the incubation time was observed. It was presumed that reconstitution of collagen fiber occurred after the dissolution of some of collagen fibers of the disc. Here, PBS means phosphate-buffered saline.

### Example 4

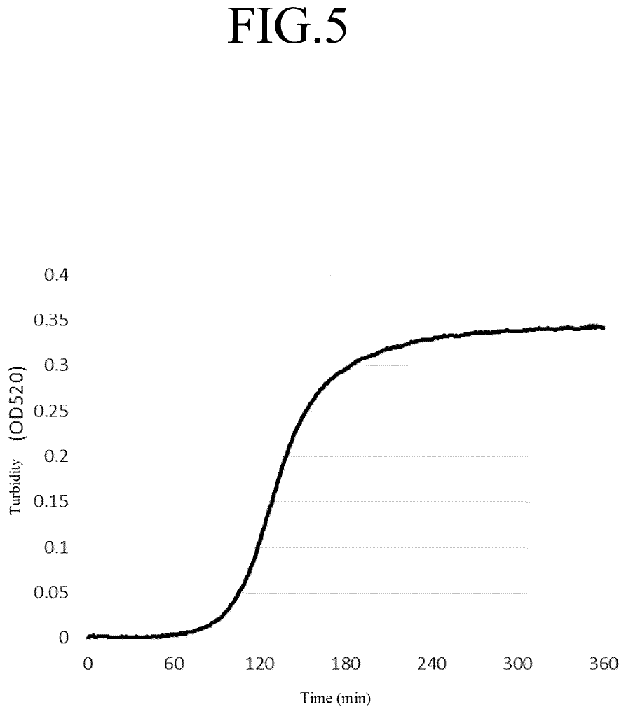

A collagen acetic acid solution was obtained by dissolving 20 mg of the oriented sheet prepared in Example 2 in 20 mL of 5 mM acetic acid. To 1 mg/mL of this acetic acid solution of collagen, the same amount of 2×PBS (2-fold concentration of PBS) as the acetic acid solution of collagen was added to adjust the collagen fiber concentration to 0.5 mg/mL and pH to 7.4, followed by incubation at 37° C. The turbidity (OD520) was measured for 360 minutes from the start of incubation. FIG. 5 illustrates the results. The collagen solution obtained by redissolving the oriented sheet showed a sharp increase in turbidity after 100 minutes of incubation, demonstrating that it has an ability to form fibrils.

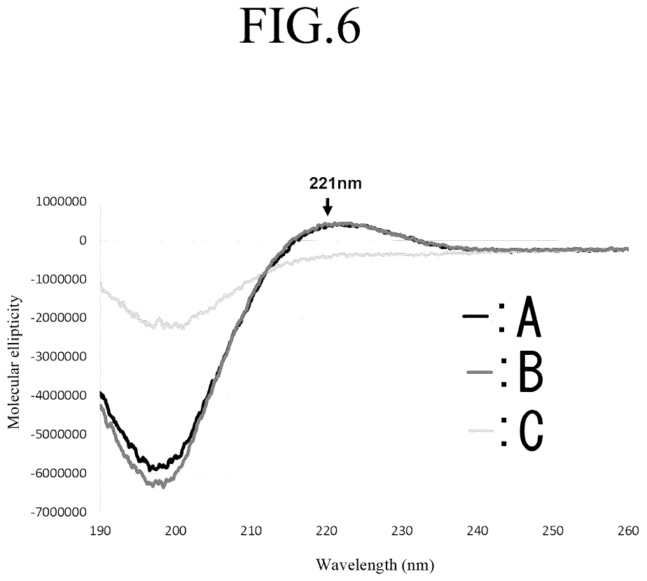

The CD spectrum of the acetic acid solution of the oriented sheet-derived collagen prepared above was measured at 20° C. using a circular dichroism spectrometer (JASCO Corporation: J-805). A collagen powder-derived acetic acid solution (collagen fiber concentration: 0.5 mg/mL) obtained by dissolving the collagen powder (PSC powder manufactured by Nippi, Incorporated) used in Example 1 in 20 mL of 5 mM acetic acid was used as a control. In addition, the acetic acid solution of the oriented sheet-derived collagen prepared above was heat-denatured at 50° C. for 5 minutes and the CD spectrum of the heat-denatured acetic acid solution was measured at 20° C. using a circular dichroism spectrometer (JASCO Corporation: J-805) in the same manner as above. FIG. 6 illustrates the results. The CD spectra of the acetic acid solution of oriented sheet-derived collagen are indicated by A, the acetic acid solution of collagen powder is indicated by B, and the acetic acid solution of oriented sheet-derived collagen heat-denatured at 50° C. is indicated by C. There was a 221-nm peak indicating the undenatured state for the acetic acid solution of oriented sheet-derived collagen (A) and the acetic acid solution of collagen powder-derived collagen (B). Meanwhile, 221-nm peak indicating the undenatured state disappeared for the heat-denatured acetic acid solution of oriented sheet-derived collagen (C), showing that heating at 50° C. causes thermal denaturation of collagen.

### Example 5

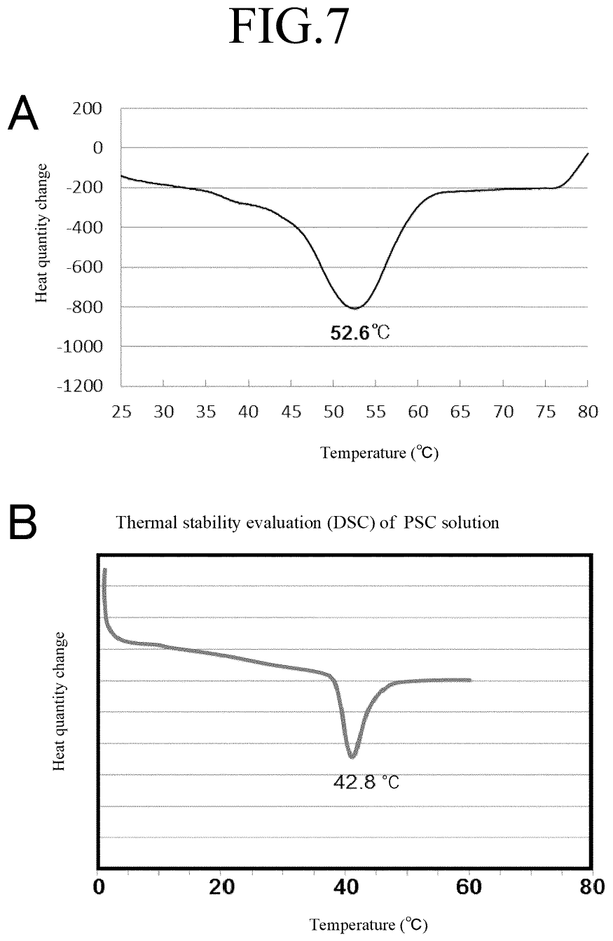

The oriented sheet prepared in Example 2 was wetted with PBS and then the denaturation temperature was measured using a differential scanning calorimeter (Seiko Instruments Inc.: DSC6100). FIG. 7 illustrates the results in panel A. In addition, the denaturation temperature was measured in the same manner for a collagen powder-derived acetic acid solution (collagen fiber concentration: 0.5 mg/mL) obtained by dissolving the collagen powder (PSC powder manufactured by Nippi, Incorporated) used in Example 1 in 20 mL of 5 mM acetic acid, as the control. FIG. 7 illustrates the results in panel B. The oriented sheet wetted with PBS showed a peak of significant heat quantity change at 52.6° C., but the denaturation temperature of the acetic acid solution of collagen was 42.8° C. Since the peak of heat quantity change is more significant for the oriented sheet than for the acetic acid solution of collagen, it was found that the oriented sheet wetted with PBS has higher thermal stability than the acetic acid solution of collagen.

### Example 6

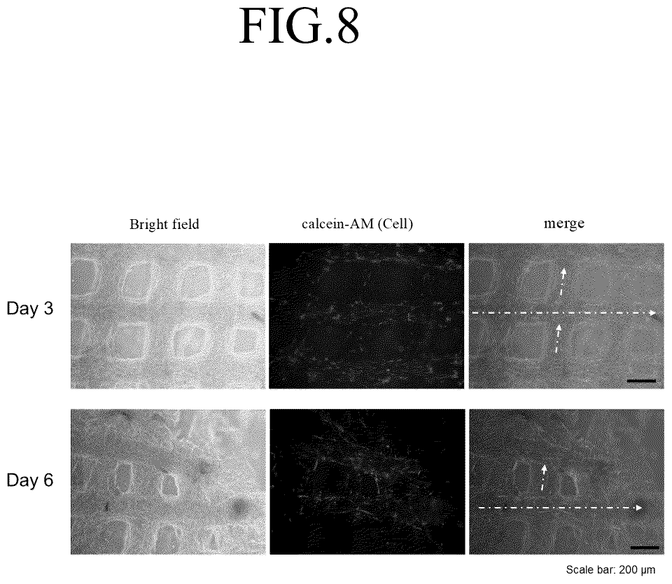

The grid-like sheet prepared in Example 2 was wetted with a DMEM containing 10% FBS. Then, fibroblasts (human embryonic lung-derived fibroblasts) were seeded on the surface of the grid-like sheet at 1×10⁴ cells/cm², followed by static culture at 37° C. The dish surface was previously coated with synthetic phospholipid (NOF CORPORATION: LIPIDURE (registered trademark)) so as to prevent cells from adhering to the dish surface. FIG. 8 illustrates phase-contrast microscope images (bright field), fluorescence microscope images after calcein staining, and merged images on days 3 and 6 of culture. The cells already adhered and spread along the grid on day 3 of culture. Similar results were obtained on day 6 of culture, and no collapse of the grid-like sheet was detected.

### Example 7

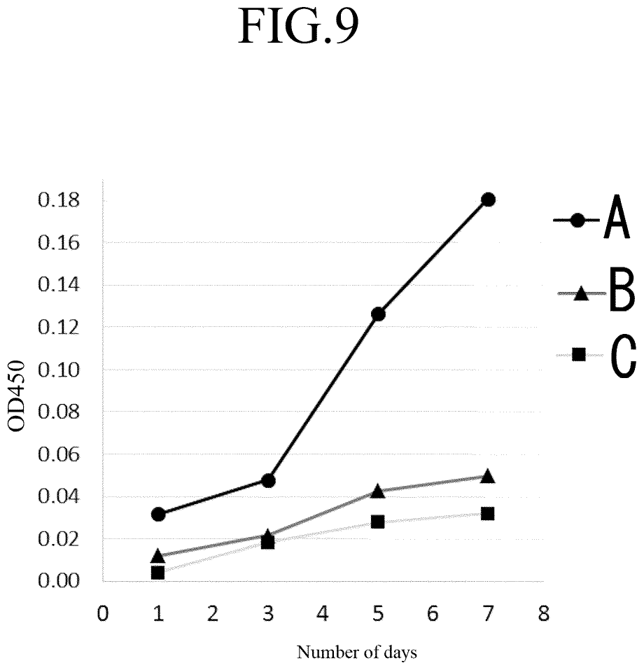

The oriented sheet prepared in Example 2 was wetted with DMEM containing 10% FBS as in Example 6, thereby preparing a DMEM-wetted oriented sheet. The oriented sheet was statically placed in a dish and fibroblasts were seeded on the surface of the oriented sheet at 1×10⁴ cells/cm², followed by static culture at 37° C. for 7 days. The dish surface was previously coated with synthetic phospholipid (NOF CORPORATION: LIPIDURE (registered trademark)) so as to prevent cells from adhering to the dish surface. An acid soluble collagen (manufactured by Nippi, Incorporated; acetic acid solution with a concentration of 50 μg/mL) coated dish at 16.7 μg/cm² and collagen gel with a concentration of 1 mg/mL were used as controls, and cells were seeded on each thereof at 1×10⁴ cells/cm². The cell growth rate was evaluated using Cell Counting Kit-8 (manufactured by DOJINDO LABORATORIES) based on the change in OD450 of the culture medium. FIG. 9 illustrates the results. In FIG. 9, 16.7 μg/cm² of the acid soluble collagen coated-dish are indicated by A, 1 mg/mL of collagen gel are indicated by B, and the DMEM-wetted oriented sheet are indicated by C. The culture using the DMEM-wetted oriented sheet (C) showed that compared to the culture using the collagen coated-dish (A), the turbidity of the medium was low during the incubation time, and cell growth was similar to that of collagen gel culture. Since collagen gel consists of a collagen fiber close to that of a living body, it was thought that the DMEM-wetted oriented sheet also has an environment close to that of a living body.

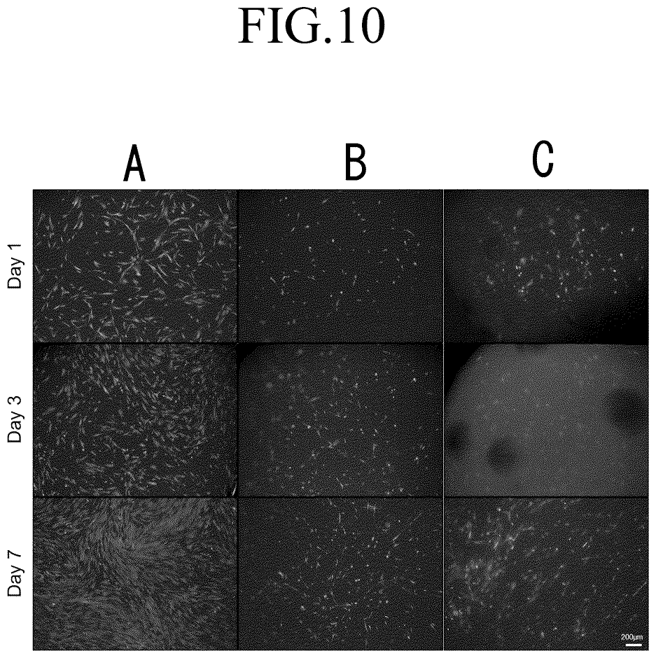

Calcein staining was performed on days 1, 3, and 7 of culture, and a fluorescence microscope image was taken each day. FIG. 10 illustrates the results. Remarkably increased cells were observed on the acid soluble collagen coated-dish (A). Meanwhile, similar to the collagen gel (B), moderate cell growth was confirmed on the DMEM-wetted oriented sheet (C). It was considered that such moderate cell growth reflected an environment similar to the in vivo.

### Example 8

As in Example 1, a collagen powder (PSC powder manufactured by Nippi, Incorporated) with an average fiber length of 142 μm and deionized water at 4° C. were mixed, thereby preparing bioinks having collagen fiber concentrations of 10%, 20%, and 30% (w/w). The bioinks were incubated at three temperature conditions of 4° C., 25° C., and 37° C. for 24 hours, and the change in collagen fiber length was measured. Measurement was carried out by a method in which the average fiber length is obtained by taking a portion of each bioink, dispersing in a 50 mM Tris-HCl buffer (pH 8.0), randomly selecting 20 or more collagen fibers with a phase contrast microscope, measuring the fiber length, and calculating the average of 20 or more collagen fibers. The results are listed in Table 1 below. There was no significant change in the average fiber length of collagen fibers over 24 hours for any bioink, confirming that it was stable. As a control, the raw material, PSC powder, was dispersed in a 50 mM Tris-HCl buffer (pH 8.0), and the average fiber length was measured in the same manner as above and found to be 151 μm.

TABLE 1

| Collagen concentration | Average fiber length of collagen (μm) | | |
|---|---|---|---|
| (w/w %) | 4° C. | 25° C. | 37° C. |
| 10 | 151 | 158 | 158 |
| 20 | 155 | 156 | 155 |
| 30 | 154 | 156 | 157 |

### Example 9

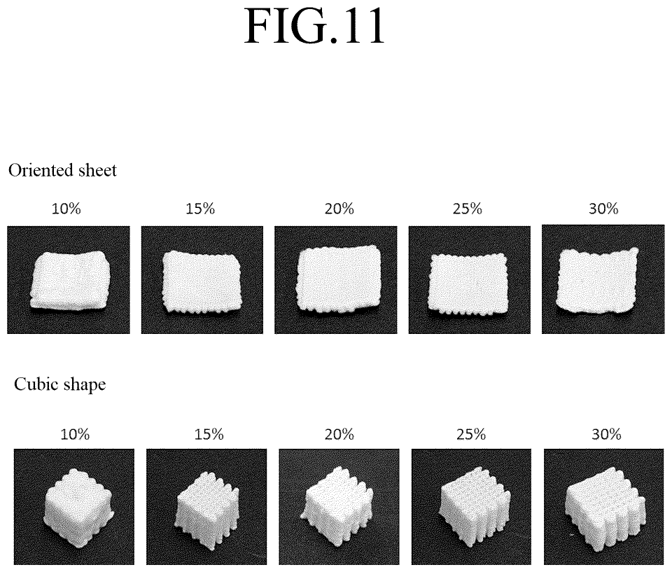

As in Example 1, a collagen powder (PSC powder manufactured by Nippi, Incorporated) with an average fiber length of 142 μm and deionized water at 4° C. were mixed, thereby preparing bioinks having collagen fiber concentrations of 5%, 10%, 15%, 20%, 25%, and 30% (w/w). The bioinks were used and were extruded by applying pressure at 25° C. via a nozzle with an inner diameter of 0.4 mm or 0.8 mm from the same dispensing 3D printer (Musashi Engineering, Inc., SHOT mini, Model M22-123) as in Example 2 into the atmosphere, thereby forming an oriented sheet with a length of 1 cm×a width of 1 cm and a grid-like cube with a length of 1 cm×a width of 1 cm×and height of 1 cm, followed by freeze-drying. The nozzle with an inner diameter of 0.8 mm was exclusively used for a product having collagen fiber concentration of 30% (w/w), and the nozzle with an inner diameter of 0.4 mm was used for the bioinks having the other concentrations. FIG. 11 illustrates the 10% (w/w) to 30% (w/w) 3D shaped objects. The 5% (w/w) and 10% (w/w) bioinks were slightly collapsed in the layer formation step, but the bioinks could be smoothly extruded from the nozzle into the atmosphere. Meanwhile, at collagen fiber concentrations of 15 to 30% (w/w), the bioinks could be smoothly extruded from the nozzle into the atmosphere, and a shaped object that did not collapse even in the layer formation step could be produced. Arbitrary five or more lines were selected from the lines forming the grid-like cubes obtained from bioinks having various collagen concentrations, their line widths were measured, and the average line width was defined as the resolution. At 5% (w/w) and 10% (w/w), the printed products collapsed, and thus the resolution was not measured. Meanwhile, the resolutions of the bioinks at 15% (w/w), 20% (w/w), 25% (w/w), and 30% (w/w) were 478.0 μm, 471.2 μm, 488.1 μm, and 813.6 μm, respectively. A high resolution of about 480 μm could be ensured for the shaped object formed with the 0.4-mm nozzle at collagen fiber concentrations of 15 to 25%. In addition, the resolution of the shaped object formed with the 0.8-mm nozzle was about 810 μm at a collagen concentration of 30% (w/w). Thus, a high resolution corresponding to the nozzle diameter could be secured.

### Comparative Example 1

A porcine dermis-derived collagen solution solubilized with pepsin was adjusted to pH 8.0 with a phosphate buffer and allowed to stand still for isoelectric precipitation, thereby obtaining an isoelectrically-precipitated collagen with an average collagen fiber length of about 1,260 μm. This precipitated collagen was dispersed in deionized water, thereby preparing bioinks having collagen fiber concentrations of 5%, 10%, 15%, and 20% (w/w). As in Example 8, the bioinks were extruded by applying pressure via a nozzle with a diameter of 0.4 mm from a dispensing 3D printer (Musashi Engineering, Inc., SHOT mini, Model M22-123), thereby making attempts to form grid-like cubes. However, within a collagen fiber concentration range of from 5 to 20% (w/w), each bioink clogged the nozzles, making it impossible to perform stable extrusion and to produce a shaped object. It was considered that the collagen fiber length was long, causing the clogging of the nozzle.

### Comparative Example 2

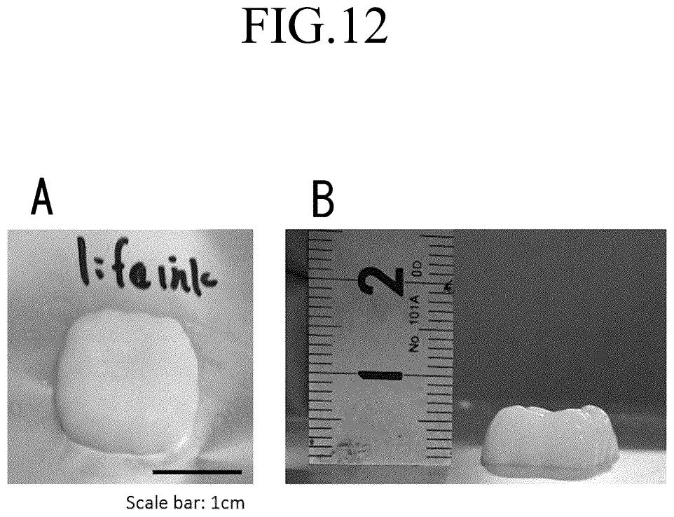

The average fiber length of collagen fibers of Lifeink (registered trademark) 200 (Neutralized Type I Collagen Bioink, 35 mg/mL, Catalog #5278) manufactured by Advanced BioMatrix was measured using a phase contrast microscopy as described in Example 1, resulting in 1,933 μm. This was used as a material and extruded by applying pressure via a nozzle with a diameter of 0.4 mm from a dispensing 3D printer (Musashi Engineering, Inc., SHOT mini, Model M22-123) into the atmosphere as in Example 2, thereby forming a grid-like cube with a length of 1 cm×a width of 1 cm×and a height of 0.6 cm at 25° C. The viscosity of the ink was very low, and even when the line width was set to 0.5 mm, it was extruded with a line width of 1 mm or more. As a result, it was difficult to produce a structure with a 3D printer. In addition, the ink was merely formed into layers, and a grid shape could not be formed. FIG. 12 illustrates this formed product. Panel A is a top view image thereof and panel B is a side view image thereof. The resolution could not be measured.

### Comparative Example 3

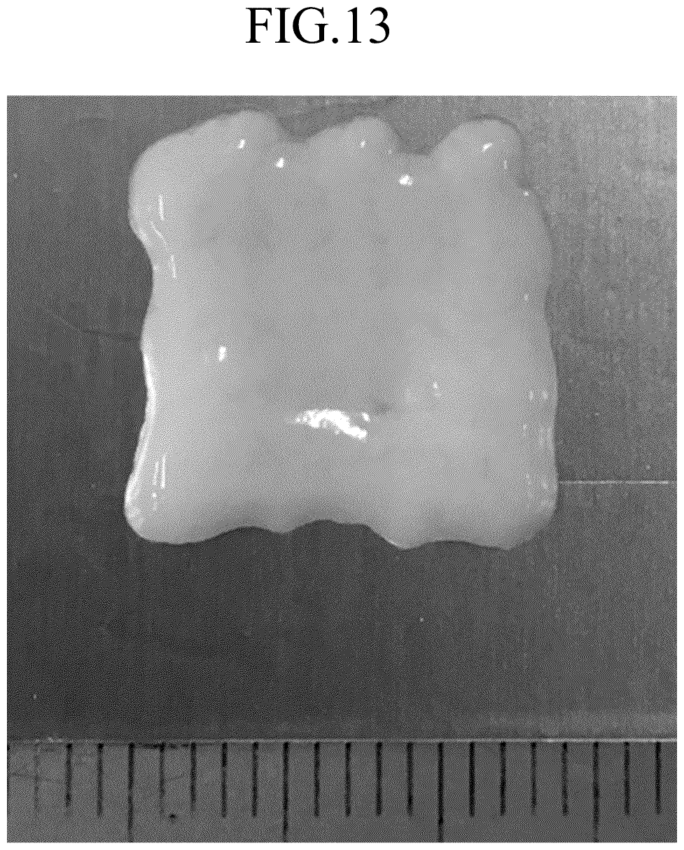

Lifeink (registered trademark) 200 (Neutralized Type I Collagen Bioink, 35 mg/mL, Catalog #5278) manufactured by Advanced BioMatrix used in Comparative Example 2 was used as a material and extruded via a 30 G needle (NIPRO CORPORATION, Flow Max 30 G×½) from a 3D printer (Musashi Engineering, Inc., SHOT mini) into the atmosphere at an air pressure of 353 kPa, thereby obtaining a grid-like cube with a length of 1 cm×a width of 1 cm×and a height of 0.6 cm. Although the 30 G needle has a small diameter of 0.12 mm in inner diameter, it was difficult to form layers, making it impossible to create a grid-like structure. FIG. 13 illustrates this formed product.

### Example 10

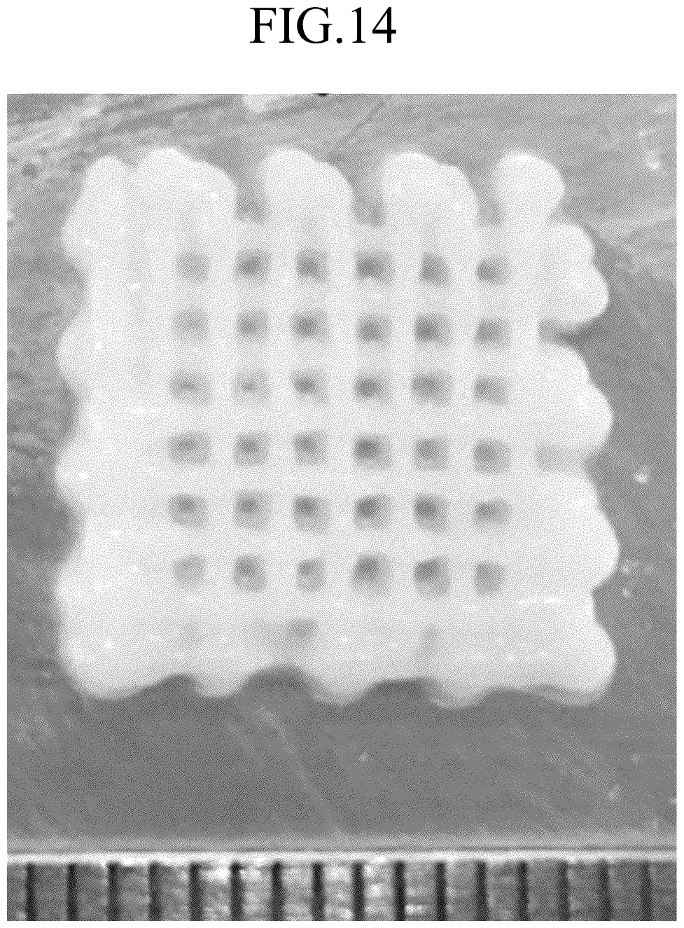

An isoelectric precipitate of chicken-derived type II collagen extracted by enzymatic treatment with proctase instead of pepsin was dried at a degree of vacuum of 40 torr and a drying temperature of 40° C. for 4 hours using a vibration dryer VU-45 manufactured by CHUO KAKOHKI CO., LTD., thereby obtaining a collagen powder. The average fiber length was 133 μm. The same operation as in Example 1 was performed using this collagen powder, thereby preparing a bioink with a collagen fiber concentration of 20% (w/w). The same operation as in Example 2 was performed using this bioink to extrude the bioink into the atmosphere at an air pressure of 59 kPa, thereby producing a grid-like sheet 1 cm (length)×1 cm (width)×0.1 cm (height) in size. FIG. 14 illustrates this shaped object. The resolution of this grid-like sheet was 466 μm. Even using the chicken-derived type II collagen, a grid-like sheet with excellent resolution could be produced.

### Example 11

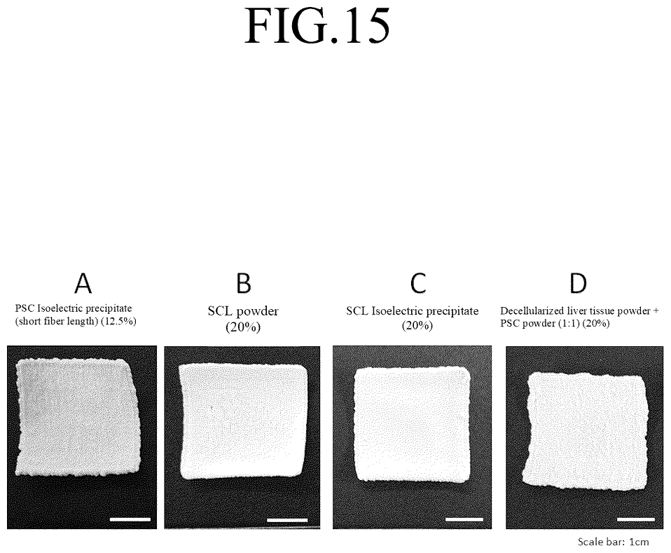

Instead of the bioink containing a collagen powder (PSC powder manufactured by Nippi, Incorporated) with an average fiber length of 142 μm used in Example 1, a bioink (A) obtained by adding deionized water to an isoelectric precipitate of porcine dermis-derived pepsin-solubilized collagen with a collagen fiber length of about 131 μm obtained by multiple centrifugal concentration and adjusting the collagen fiber concentration to 12.5% (w/w), a bioink (B) containing a porcine dermis-derived alkali-solubilized collagen powder with a collagen fiber length of about 158 μm at 20% (w/w) in deionized water, a bioink (C) obtained by adding deionized water to an isoelectric precipitate of porcine dermis-derived alkali-solubilized collagen with a collagen fiber length of about 141 μm obtained by multiple centrifugal concentration and adjusting the collagen fiber concentration to 20% (w/w), and a bioink (D) with a collagen fiber concentration of 20% (w/w) prepared by mixing the PSC powder used in Example 1 and a decellularized liver tissue powder fractionated with a 100- to 250-μm sieve at an equal weight ratio (1:1) were prepared. These bioinks were used and extruded via a nozzle with a diameter of 0.4 mm from a dispensing 3D printer into the atmosphere by applying pressure as in Example 2, thereby printing each bioink into a sheet form. The obtained printed products were freeze-dried. FIG. 15 illustrates freeze-dried printed products. As illustrated in FIG. 15, regardless of the solubilization method of collagen constituting a collagen fiber or the preparation method of a bioink, a 3D shaped object could be produced using a 3D printer, even in a case in which other components were added to collagen fibers.

### Example 12

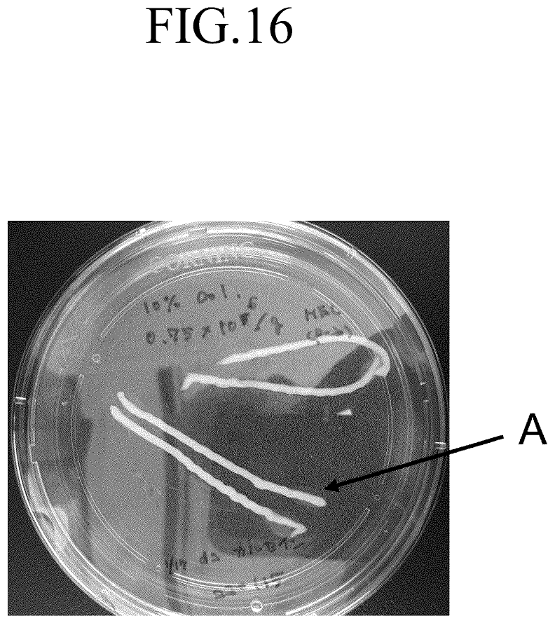

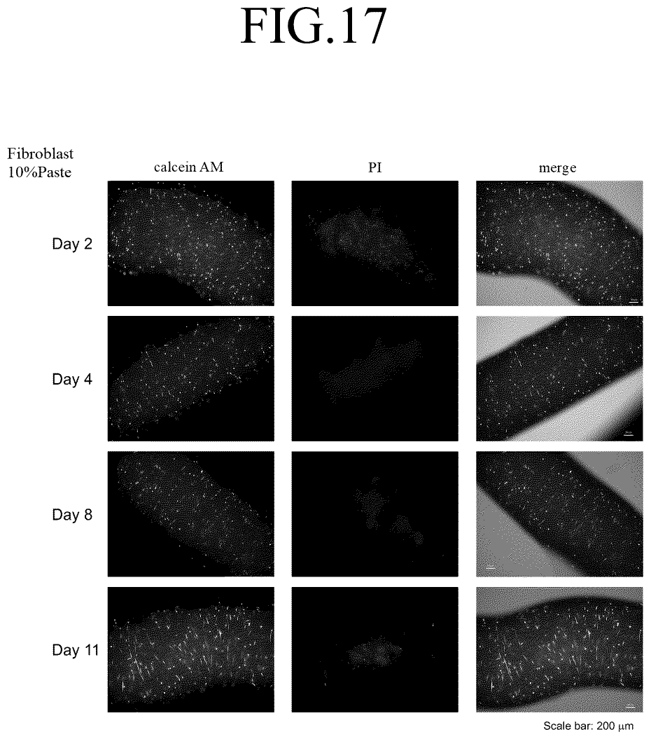

To 0.2 g of the collagen powder (PSC powder manufactured by Nippi, Incorporated) with an average fiber length of 142 μm used in Example 1, 1.8 mL of a 10% FBS/DMEM containing fibroblasts (human embryonic lung-derived fibroblasts) at 0.75×10⁶ cells/mL was added, and lightly mixed using a dispensing spoon, thereby preparing a cell-laden bioink with a collagen fiber concentration of 10% (w/w). This cell-laden bioink was filled into a 10-mL syringe and extruded via an 18 G (gauge) needle into a container. The collagen powder in the cell-laden bioink was uniformly mixed. Subsequently, the further extruded cell-laden bioink was mixed at 25° C. and 1,000 rpm for 1 minute with a planetary centrifugal mixer (Awatori Rentaro manufactured by THINKY CORPORATION). This cell-laden bioink was filled into a 10-ml syringe and extruded via an 18 G (gauge) needle onto a dish with a diameter of 10 cm, and then mL of DMEM containing 10% FBS was added, followed by incubation at 37° C. FIG. 16 illustrates the cell-laden bioink A before incubation. Further, calcein staining (calcein AM, 1 μg/mL) and propidium iodide staining (PI, 1 μg/mL) were performed on 10 days 2, 4, 8, and 11 from the start of static culture, and fluorescence microscope images of each staining were taken. FIG. 17 illustrates fluorescence microscope images after calcein staining and those after propidium iodide staining on days 2, 4, 8, and 11 of the culture and their merged images with the bright field image. Calcein staining stains living cells, and propidium iodide staining stains dead cells. It was confirmed that the number of living cells increased, and the number of dead cells decreased with the passage of days. Thus, it was confirmed that a cell-embedded 3D shaped object can be produced using a cell-laden bioink. As the dish, a dish that had been treated with LIPIDURE (registered trademark) (NOF CORPORATION) such that cells did not adhere to the surface of the dish was used.

### Example 13

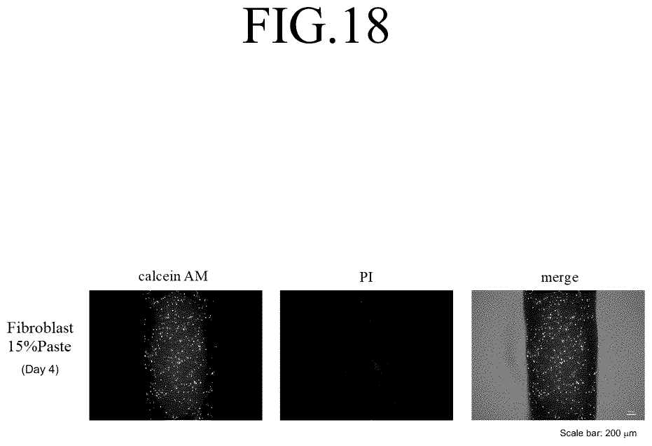

To 0.3 g of the collagen powder (PSC powder manufactured by Nippi, Incorporated) used in Example 12, 1.7 mL of a 10% FBS/DMEM containing fibroblasts (human embryonic lung-derived fibroblasts) at 1×10⁶ cells/mL was added, and lightly mixed using a dispensing spoon, thereby preparing a cell-laden bioink with a collagen fiber concentration of 15% (w/w) which was higher than that in Example 12. This cell-laden bioink was filled into a 10-mL syringe and extruded via an 18 G (gauge) needle into a container. The collagen powder in the cell-laden bioink was uniformly mixed. Subsequently, the further extruded cell-laden bioink was mixed at 25° C. and 1,000 rpm for 1 minute with a planetary centrifugal mixer (Awatori Rentaro manufactured by THINKY CORPORATION). This mixed cell-laden bioink was filled into a 10-mL syringe and extruded via an 18 G (gauge) needle onto a LIPIDURE (registered trademark)-treated dish with a diameter of 10 cm, and then 10 mL of DMEM containing 10% FBS was added, followed by incubation at 37° C. Further, calcein staining (calcein AM, 1 μg/mL) and propidium iodide staining (PI, 1 μg/mL) were performed on day 4 from the start of static culture, and fluorescence microscope images of each staining were taken. FIG. 18 illustrates fluorescence microscope images after calcein staining and those after propidium iodide staining on day 4 of the culture and their merged images with the bright field image. It was confirmed that there were more living cells and fewer dead cells than in Example 12 on day 4 of static culture.

### Example 14

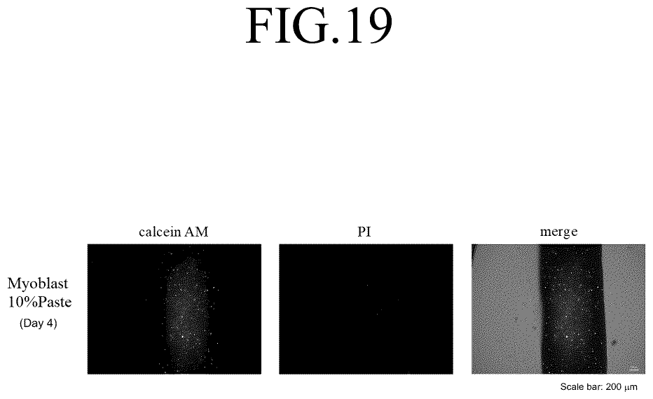

To 0.2 g of the collagen powder (PSC powder manufactured by Nippi, Incorporated) used in Example 12, 1.8 mL of a 20% FBS/DMEM containing myoblasts (mouse myoblasts, C2C12) at 1.4×10⁶ cells/mL, different from the fibroblasts used in Example 12, was added, and lightly mixed using a dispensing spoon, thereby preparing a cell-laden bioink with a collagen fiber concentration of 10% (w/w). This cell-laden bioink was filled into a 10-mL syringe and extruded via an 18 G (gauge) needle into a container. The collagen powder in the cell-laden bioink was uniformly mixed. Subsequently, the further extruded cell-laden bioink was mixed at 25° C. and 1,000 rpm for 1 minute with a planetary centrifugal mixer (Awatori Rentaro manufactured by THINKY CORPORATION). This mixed cell-laden bioink was filled into a 20-mL syringe and extruded via an 18 G (gauge) needle onto a LIPIDURE (registered trademark)-treated dish with a diameter of 10 cm, and then 10 mL of DMEM containing 20% FBS was added, followed by incubation at 37° C. Calcein staining (calcein AM, 1 μg/mL) and propidium iodide staining (PI, 1 μg/mL) were performed on day 4 from the start of static culture, and fluorescence microscope images of each staining were taken. FIG. 19 illustrates fluorescence microscope images after calcein staining and those after propidium iodide staining on day 4 of the culture and their merged images with the bright field image. The presence of living cells was also confirmed in the myoblasts-laden bioinks. Thus, it was confirmed that a cell-embedded 3D shaped object can be produced using myoblasts-laden bioink.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

This application claims the benefit of Japanese Patent Application No. 2021-022602, filed on Feb. 16, 2021, the entire disclosure of which is incorporated by reference herein.

1. A bioink for use in bioprinting, comprising a collagen fiber composed of collagen and/or a collagen derivative and a solvent, wherein an average fiber length of the collagen fiber is from 0.5 to 1,000 μm.

2. The bioink according to claim 1, wherein a concentration of the collagen fiber in the solvent is from 5 to 30% (w/w).

3. The bioink according to claim 1, wherein the solvent is one or more selected from the group consisting of deionized water, a buffer, physiological saline, and a cell culture medium.

4. The bioink according to claim 1, further containing one or more compounds selected from the group consisting of an extracellular matrix molecule, a decellularized tissue, a growth factor, a cytokine, and a cross-linker.

5. The bioink according to claim 1, further containing cells.

6. The bioink according to claim 1, wherein the bioink is a bioink for a 3D printer.

7. A shaped object, comprising:
the bioink according to claim 1.

8. A crosslinked shaped object, wherein the shaped object according to claim 7 is crosslinked with one or more selected from the group consisting of riboflavin, methacrylated gelatin (GelMA), polyethylene glycol diacrylate (PEGDA), glutaraldehyde, formaldehyde, genipin, an ammonium derivative, a photoinitiator, Irgacure (registered trademark), lithium phenyl-2,4,6-trimethylbenzoylphosphinate, and ruthenium.

9. One or more products composed of the shaped object according to claim 7, selected from the group consisting of a cell culture substrate, a substrate for transplantation, a tissue construct, an organ model, and a substrate for regenerative medicine.

10. A method for producing a shaped object, the method comprising:
extruding the bioink according to claim 1 into an atmosphere by pressurization via a nozzle with a diameter of from 0.2 to 1 mm.

11. One or more products composed of the crosslinked shaped object according to claim 8, selected from the group consisting of a cell culture substrate, a substrate for transplantation, a tissue construct, an organ model, and a substrate for regenerative medicine.
